(19) United States
(12) Patent Application Publication
(10) Pub. No.: US 2008/0125716 A1
(43) Pub. Date: May 29, 2008

(54) Surgical access port sealing assembly

(76) Inventor: Lawrence Cruz, Bethel, CT (US)

Correspondence Address: Lawrence Cruz, 12 Goodhill Rd., Bethel, CT, 06801, US

(21) Appl. No.: 11/510,989

(22) Filed: Aug. 28, 2006

(51) Int. Cl. A61M 39/06

(52) U.S. Cl. 604/167.06

(57) ABSTRACT

A surgical system assembly (1) includes a resilient seal (6) having a central opening (7) therein and being fixedly mounted to a housing (2); and a non-elastic, flexible member (9) joining the housing (2) to a rigid or semi-rigid cannula (14) or tube that is positioned at least partially through tissue, percutaneously, during a surgical procedure. Flexing of the non-elastic, flexible member (9) facilitates movement of the housing (2) and thus the resilient seal (6) relative to the cannula (14) in order to allow the seal (6) to move with an instrument or object protruding therethrough in sealing engagement as a means of reducing stress concentrations that tend to promote overstretching or “cat-eyeing” of the seal's central opening (7).

### RELATED APPLICATIONS

None.

### TECHNICAL FIELD

The present invention relates to surgical access ports and, more particularly, to sealing assemblies associated with surgical access ports that enable consecutive insertion, operation and removal of one or more various instruments or objects during a surgical procedure.

### BACKGROUND OF THE INVENTION

Certain surgical procedures involve the use of tube-like or cannula shaped access ports (210), such as shown in FIGS. 1A-1F, inserted into the body percutaneously through a relatively small incision. Such devices are commonly used in procedures where the abdominal region is insufflated so that using a plurality of access ports a surgeon can perform procedures while having an optical device inserted. Known cannula shaped devices for such use are typically coupled with an elongated rod having a tapered end and a small blade at the tip, the device sometimes being called an obturator or a trocar blade. The trocar blade is nested in the cannula (212) and the assembly is pushed through the skin at the site of the small incision. Then the trocar blade is removed and the cannula (12) is left in place forming an access port. One or more access ports are put into place and if desired pressurized gas is introduced to insufflate the abdomen, and various instruments and objects can be passed into and out of the body through the ports to perform specific procedures.

Located at the upper end of the cannula is a sealing assembly (214). The assembly contains a seal (216) that is designed to seal around the circumference of an instrument shaft (218) while preventing or limiting the escape of pressurized gas or liquid from an insulated region through the cannula (212). The seal (216) typically has a round opening (220) that will accommodate shaft diameters ranging from 4 mm to 15 mm, as well as objects of varying shapes and sizes.

A secondary seal (222) is located down inside the cannula (212) toward its distal end. Its function is to seal and prevent or limit pressurized gas and liquid escape when no instrument or object is spanning the first seal opening (220). Common secondary seal designs are either “duckbill” or “flapper” styles that default to a closed position by back pressure, spring bias, or both.

A common shortcoming of such seal assemblies is that, most often with smaller diameter instrument shafts, lateral movement of such shafts causes the opening in the seal (220) to stretch into a non-round shape, such as a “cat eye” shape, as illustrated in FIG. 1F, thereby enabling the undesirable escape of pressurized insufflation gas adjacent to the shaft (218).

Certain existing seal designs have been introduced in order to remedy the cat-eyeing problem, resulting in undesirably complex, inefficient, costly or poorly performing devices. Such designs are shown in U.S. Pat. No. 5,385,553. For illustrative purposes, two prior art concepts are schematically illustrated in FIGS. 1G and 1H. Referring to FIG. 1G, an elastomeric seal (224) is mounted to a rigid ring (226) which is provided with flexible wipers (228, 230) that engage, respectively, the floor (232) and ceiling (234) of the interior of a seal housing on a trocar device. When an instrument shaft is positioned inside the opening (236) of the elastomer seal (224) and moved laterally, such as in the X-Z plane illustrated in FIG. 1G, the wipers (228, 230) move relative to the floor (232) and ceiling 234) while maintaining sealing engagement therewith. Another solution is shown schematically in FIG. 1H, wherein a seal comprises an elastomer first seal portion (238) mounted to a rigid ring (240) that is suspended by spring-like accordion pleats (244) of an elastic material so that when an instrument shaft is positioned inside of the seal opening (242) and moved laterally, the ring (240) and seal portion (238) move laterally in the X-Z plane. Both prior art concepts are relatively complex and costly, and require the use of resilient or elastic material in addition to the basic seal portion that surrounds and instrument shaft. Also, the prior art concept of FIG. 1G subjects lateral movement of the seal (224) to friction forces caused by the wipers (228, 230); and the prior art concept of FIG. 1H is subject to spring-like resistance caused by the pleats (244). In either case, the lateral movement of the respective seal while manipulating an instrument positioned through the respective seal is encumbered by either the friction or the spring-like resistance.

### OBJECTS OF THE PRESENT INVENTION

It is an object of the present invention to provide a seal assembly for a surgical access port such as a trocar access port that accommodates instruments and objects in a sealing manner and that avoids shortcomings such as those described above with respect to the prior art such as “cat-eyeing” or stretching associated with smaller diameter instrument shafts that results in pressure leakage adjacent the shaft. It is a further object of the present invention to provide a seal assembly that utilizes a non-elastic material to join a seal housing to a cannula to facilitate movement of the seal housing relative to the cannula in a low-resistance manner. It is a further object of the present invention to provide a seal assembly that utilizes a non-elastic material as a component in order to achieve durability, ease of assembly, and cost-efficiency. These an other objects are achieved by the present invention disclosed herein.

### SUMMARY OF THE PRESENT INVENTION

A surgical system assembly according to the present invention includes a resilient seal having a central opening therein and fixedly mounted to a housing; and a non-elastic, flexible member joining the housing to a rigid or semi-rigid cannula or tube that is positioned at least partially through tissue, percutaneously, during a surgical procedure. Flexing of the non-elastic, flexible member facilitates movement of the housing and thus the resilient seal relative to the cannula in order to allow the seal to move with an instrument or object protruding therethrough in sealing engagement as a means of reducing stress concentrations that tend to promote overstretching or “cat-eyeing”of the seal's central opening.

### BRIEF DESCRIPTION OF THE DRAWINGS

FIG. 1A is a front view of a seal assembly according to the prior art.

FIG. 1B is a cross-sectional, schematic view of a seal assembly according to the prior art.

FIG. 1C is a cross-sectional, schematic view of a seal assembly according to the prior art showing an instrument shaft inserted therein.

FIG. 1D is a bottom, schematic view of a component of a seal assembly according to the prior art.

FIG. 1E is a partial, front schematic view of a seal assembly according to the prior art showing an instrument inserted therein.

FIG. 1F is a partial, front schematic view of a seal assembly according to the prior art showing an instrument inserted therein and positioned laterally.

FIG. 1G is a cross-sectional, schematic view of a seal assembly according to the prior art.

FIG. 1H is a cross-sectional, schematic view of a seal assembly according to the prior art.

FIG. 2A is a front, schematic view of a seal assembly according to a first embodiment of the present invention.

FIG. 2B is a cross-sectional, schematic view of a seal assembly according to a first embodiment of the present invention.

FIG. 2C is a top view of a component of a seal assembly according to a first embodiment of the present invention.

FIG. 3 is a cross-sectional, schematic view of a seal assembly according to a another embodiment of the present invention.

FIG. 4A is a cross-sectional, schematic view of a seal assembly according to a another embodiment of the present invention.

FIG. 4B is a front perspective view of a component of the assembly according to FIG. 4A.

FIG. 5 is a partial cross-sectional, schematic view of a seal assembly according to a another embodiment of the present invention.

FIG. 6 is a partial cross-sectional, schematic view of a seal assembly according to a another embodiment of the present invention.

FIG. 7A is a cross-sectional, schematic view of a seal assembly according to a another embodiment of the present invention.

FIG. 7B is a cross-sectional, schematic view of a seal assembly according to a another embodiment of the present invention.

FIG. 7C is a partial, schematic top view of an optional component according to the embodiment of FIG. 7B.

FIG. 7D is a cross-sectional, schematic view of a seal assembly according to a another embodiment of the present invention.

FIG. 7E is a schematic front perspective view of a component according to the embodiment of FIG. 7D.

FIG. 7F is a cross-sectional, schematic view of a seal assembly according to a another embodiment of the present invention.

FIG. 7G is a schematic, front perspective view of a component according to the embodiment of FIG. 7F.

FIG. 7H is a partial cross-sectional schematic view of an alternative construction according to the embodiment of FIG. 7F.

FIG. 7I is a partial, cross-sectional schematic view of an optional component according to the embodiment of FIG. 7F.

FIG. 8A is a cross-sectional, schematic view of a seal assembly according to a another embodiment of the present invention.

FIG. 8B is a schematic, partial cross-sectional perspective view of the embodiment of FIG. 8A.

FIG. 8C is a view according to FIG. 8A, illustrating a lateral movement condition.

FIG. 8D is a partial, schematic view of several components of the embodiment of FIG. 8A.

FIG. 8E is a front, perspective schematic view of a component according to the embodiment of FIG. 8A.

FIG. 9A is a cross-sectional, schematic view of a seal assembly according to a another embodiment of the present invention.

FIG. 9B is a front, perspective schematic view of a component according to the embodiment of FIG. 9A.

FIG. 10A is a top, schematic view of a seal assembly according to another embodiment of the present invention.

FIG. 10B is a first cross-sectional schematic view of the embodiment according to FIG. 10A.

FIG. 10C is a second cross-sectional schematic view of the embodiment according to FIG. 10A.

FIG. 11 is a cross-sectional, schematic view of a seal assembly according to a another embodiment of the present invention.

FIG. 12A is a partial cross-sectional, schematic view of a seal assembly according to a another embodiment of the present invention.

FIGS. 12B, 12C and 12D are partial cross-sectional schematic views of a component shown in various configuration in accordance with the embodiment of FIG. 12A.

FIG. 13 is a cross-sectional, schematic view of a seal assembly according to a another embodiment of the present invention.

### DETAILED DESCRIPTION OF THE PREFERRED EMBODIMENTS

As illustrated throughout the drawing figures and description for reference, the axes labeled X-Y-Z indicate directions that are generally orthogonal to each other. While the preferred embodiments of the invention are herein described with respect to commonly known trocar systems used in various surgical procedures such as laparoscopic procedures by way of example, the present invention is not limited to such uses and it may be used in a variety of different settings and for a variety of different purposes.

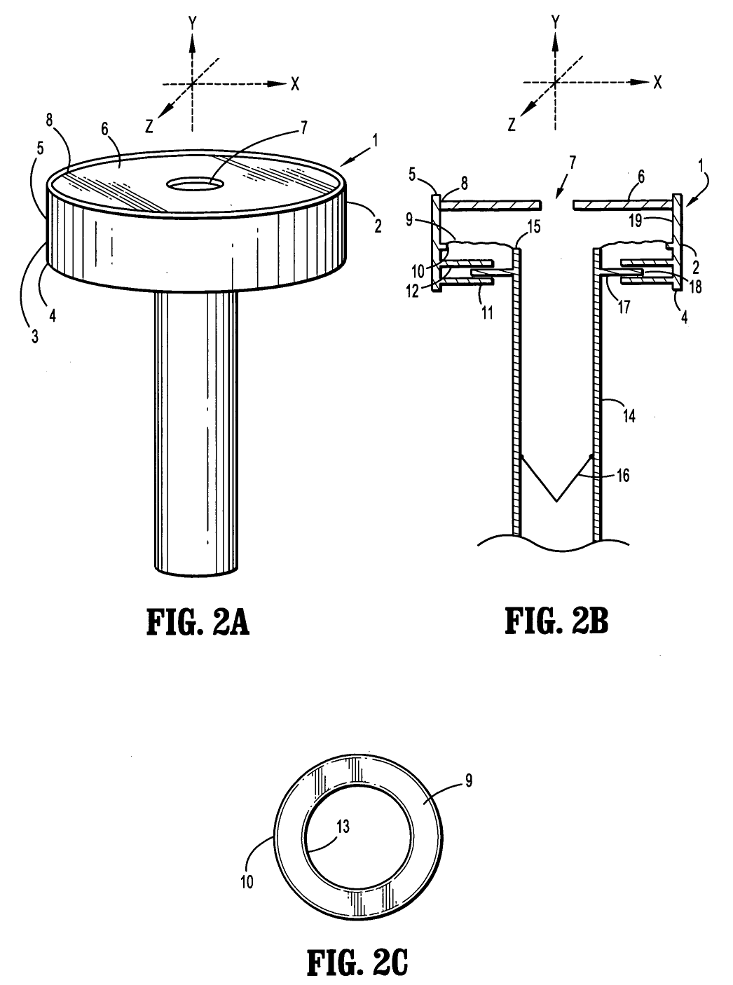

Referring to FIGS. 2A-2C, a seal assembly (1) according to a first embodiment of the present invention is housed in a trocar seal housing (2). The housing (2) is preferably round in profile and has a circular, vertical wall (3) defined by a lower edge (4) and an upper edge (5). A flexible, elastomeric seal (6) having a central opening (7) therethrough is attached at it circumferential edge (8) to the inside of the upper edge (5) of the wall (3). A non-elastic, flexible sheet (9) is attached at its outer circumferential edge (10) to the inside of the wall (3) at an intermediate location between the lower edge (4) and the upper edge (5). The sheet (9) may be made of any nor-porous material of sufficient strength to seal pressures encountered in surgical procedures such as laparoscopic procedures in which insufflation is implemented without tearing or leaking. Preferably, the sheet (9) is made from any one of a variety of known, non-elastic, plastic or polymer materials and could be made of a fabric, impregnated fabric including nylon or polyester impregnated with rubber or rubber-like materials, or a laminate comprising similar or varying materials. Desired flexibility of the sheet (9) is determined by user preference and, in a preferred mode of the present invention, maximum flexibility is desired for reasons explained below. Thus, in the preferred mode a sheet (9) having flexibility similar to or greater than that of a common household trash bag is desirable. In an alternative embodiment of the present invention, a sheet (9) having a predetermined level of porosity to allow a predetermined amount of pressure to escape at a predetermined rate may be utilized.

The housing (2) has two internal flanges, a lower flange (11) and an upper flange (12), that each are attached to the internal surface of the wall (3). The flanges (11, 12) may be flat ring shapes or they may be intermittent tabs arranged circumferentially such as in a flower petal configuration. The sheet (9) has an internal opening defined by an inner circumferential edge (13). The housing (2) is joined to an elongated, tube-shaped cannula (14) of a type resembling the cannula component of commonly known trocar cannulas. The housing (2) and cannula (14) are joined by the sheet (9) in that the inner circumferential edge (13) is attached to the upper edge (15) of the cannula (14). The sheet (9) may be joined to the cannula (14) and to the housing (2) by any one or more of known methods including, but not limited to, welding, adhesive, and molding. The cannula (14) may be provided with a secondary seal (16) of a type generally known by those skilled in the trocar system art such as a duckbill type or flapper type seal. A cannula flange (17) is provided at or near the upper edge (15) of the cannula (14). The cannula flange (17) is attached to the external circumferential wall of the cannula (14) and forms an outermost, or maximum diameter dimension at its edge (18). The maximum diameter (18) of the cannula flange (17) is less than the internal diameter of the housing (2) defined by the interior surface (19) of its wall (3).

The difference in size between the maximum diameter (18) of the cannula flange (17) and the internal diameter of the housing (2) allows relative lateral movement in the X-Z plane between the cannula (14) and the housing (2). The flexible quality of the sheet (9) permits such lateral movement. The sheet (9) is selectively oversized so that it is not in tension when the cannula (14) and housing (2) are assembled together via the sheet (9), thus forming slack or looseness. By sizing the sheet (9) accordingly, there is no need for the sheet (9) to be elastic or to have elastic properties. This is desirable since stretching to allow lateral movement, as is the case with certain prior art seal assemblies, introduces tactile resistance or force due to stretching, and could result in sudden, unintentional movement if released. Furthermore, elastic materials are more costly and more subject to wear or tearing than a non-elastic sheet such as the sheet (9) of the present invention.

The upper flange (12) and lower flange (11) are of sufficient radial length to overlap with the maximum diameter (18) of the cannula flange (17), but are not of such length as to contact the outer surface of the cannula (14). Because neither the upper and lower flanges (12, 11) nor the cannula flange (17) extend the entire distance between the cannula (14) and the interior surface (19) of the housing (2), the housing (2) has room to move laterally, in the X-Z plane, relative to the cannula (14). The overlap of the flanges (12, 11) above and below the cannula flange (17), as shown in FIG. 2B, limits or prevents undesirable vertical movement of the cannula (14) relative to the housing (2) in a vertical direction defined by the Y axis.

During use, when the distal end of the cannula (14) is percutaneously introduced into a patients body and insufflation pressure is implemented, pressure is first maintained distally of the secondary seal (16) until an object or instrument is passed through the secondary seal (16), at which time pressurized gas and/or liquid rises through the cannula (14) until it is trapped by the seal (6). When an instrument shaft (not shown) is positioned in the central opening (7), said instrument shaft being of sufficient diameter to cause the opening (7) to stretch and fit snugly, the pressurized gas cannot escape past the seal (6) or the shaft. The pressurized gas is further contained from below by the sheet (9) to prevent the gas from escaping downwardly of the sheet (9). Depending on the amount of gas pressure and the weight and configuration of the seal assembly components, the housing (2) may lift slightly and float such that the upper flange (12) will not be in contact with the cannula flange (17). The only contact between the housing (2) and the cannula (14), other than through the sheet (9), is then between the lower flange (11) and the cannula flange (17). The result is minimized friction when moving the housing (2) laterally in the X-Z plane relative to the cannula (14). If desired, enhancements can be made to minimize friction between the lower flange (11) and the cannula flange (17). For example, either flange can be lubricated. Either flange can be configured into segments rather than one continuous flange in order to reduce contact surface area and, thus, friction. Either surface can be provided with bumps or contours other than flat surfaces to minimize contact surface area at any given time. Either surface can be provided with movable bearings such as ball bearings or rotatable beads in order to minimize friction during lateral movement. Because the sheet (9) is loose fitted and does not have elastic properties, there is no return force or spring bias against lateral movement.

In each embodiment described herein, during insufflation where pressure and gas fill the interior of the cannula and the seal assembly housing, the seal assembly housing will lift or rise (Y-axis direction) relative to the cannula due to the internal pressure and, thus, will reduce friction that would otherwise inhibit lateral movement (X-Z plane). Depending on which embodiment of the present invention, upward vertical movement (Y-axis direction) will be limited be a flange, a sheet under tension, or a tether. Bearing surfaces including movable bearing elements can be provided to ease movement by further reducing friction.

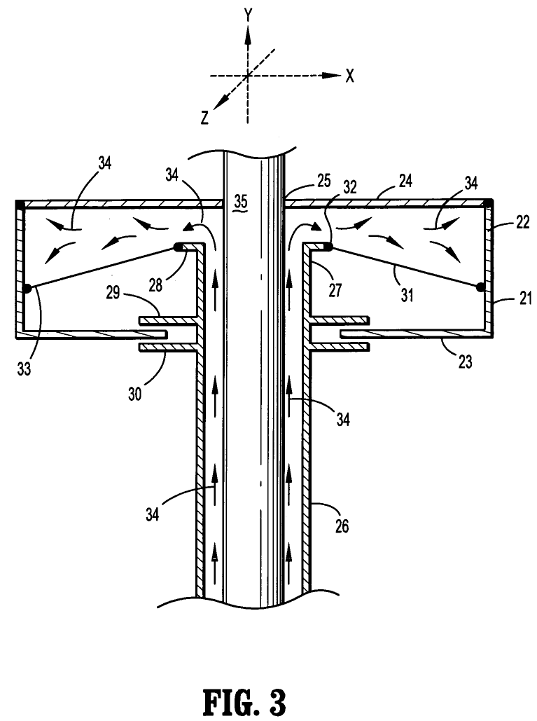

A similar embodiment is shown in FIG. 3, wherein a housing (21) comprises a vertical wall (22) and a lower flange (23). A resilient seal (24) is provided having a central opening (25) and it is attached to the wall (22) at its circumferential edge. A cannula (26) has a proximal end (27) which may have an end flange (28), and two intermediate flanges (29, 30) spaced apart vertically and capturing the lower flange (23) of the housing (21) to limit its range of vertical movement relative to the cannula (26) in the Y-direction shown. The inside diameter of the lower flange (23) is greater than that of the cannula (26) but less than the maximum diameter of the intermediate flanges (29, 30) in order to allow the housing (21) to move laterally in the X-Z plane. A non-resilient sheet (31) having a ring-shape or shaped like a cone with a central opening is provided by attaching its inside diameter edge (32) to the cannula flange (28) and by attaching its outside circumferential edge (33) to the inside of the wall (22). The sheet (31) may be slightly oversized to enable the housing (21) to move laterally (X-Z plane) and vertically (Y-axis) relative to the cannula without substantial resistance. The sheet (31) material is sufficient to seal pressures and gases used during insufflation in surgical procedures. It may be made of any suitable material such as those described above with respect to an earlier embodiment. For illustrative purposes, pressure and gas flow arrow (34) are shown in FIG. 3, indicating the path of pressure and gas during insufflation in a surgical procedure when an instrument shaft (35) is inserted into the central opening (25) and the cannula (26) is percutaneously introduced into a patient so that its upper, or proximal end is exposed and its distal end is beneath the patients skin.

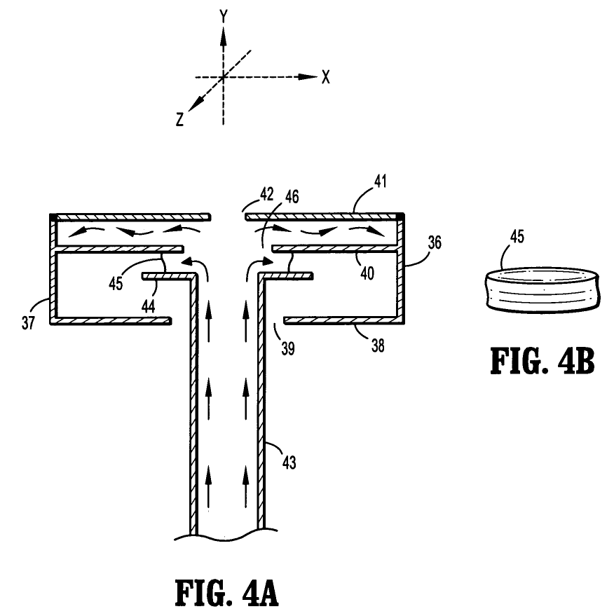

Another embodiment is shown in FIGS. 4A-4B, in which a housing (36) has a vertical wall (37), a floor (38), a central opening (39) in the floor, and in internal flange (40). A resilient seal (41) with a central opening (42) is attached within the housing (36) at its circumferential edge. A cannula (43) having a proximal end flange (44) is positioned within the central opening (39). A flexible tube of non-resilient sheet material (45) attaches the internal flange (40) of the housing to the proximal end flange (44) of the cannula (43). The outside diameter of the cannula (43) is less than the inside diameter of the central opening (39) in the floor of the housing (36) such that the housing (36) can move relative to the cannula (43) laterally, in the X-Z plane. The proximal end flange (44) of the cannula (43) is of sufficient radial dimension to engage the internal flange (40) of the housing (36) when the housing (36) moves downwardly, along the Y axis, relative to the cannula (43) and, thus, serves as a stop. The sheet tube (45) is of sufficient size and dimension to permit predetermined lateral movement (X-Z plane) of the housing (36) relate to the cannula (43), and to limit upward vertical movement (Yes) of the housing (36) relative to the cannula (43) by becoming tensioned when fully extended. The inside diameter formed by the opening (46) in the center of the internal flange (40) is selected to permit passage of instruments and objects during a surgical procedure and, thus, should be at least as large as the internal diameter of the cannula (43).

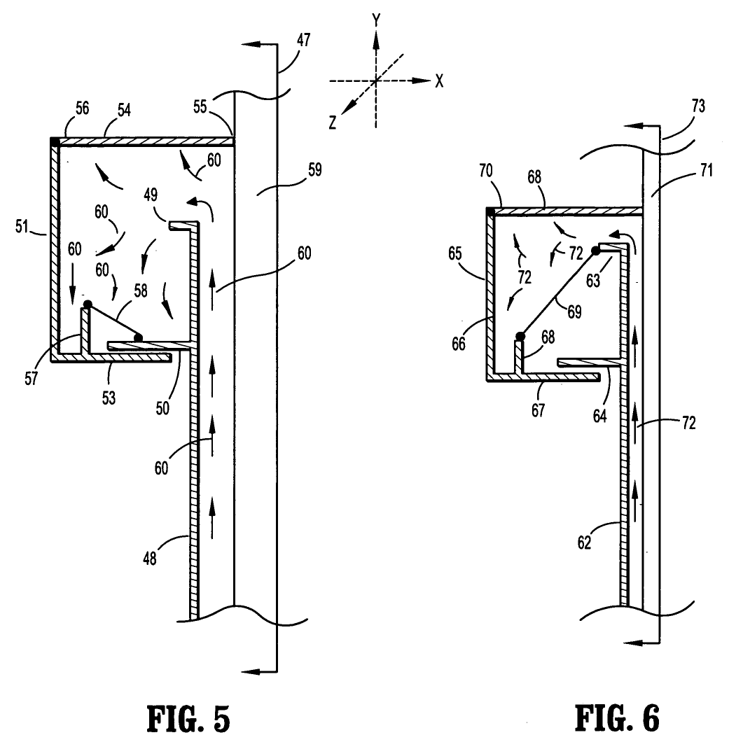

FIG. 5 illustrates another embodiment of the present invention, in partial form for clarity since the features not shown in this view are essentially similar to those of previous embodiments. For illustration, a drawing line (47) bisects the assembly. A cannula (48) has a proximal end (49) and a flange (50). A seal housing (51) has a vertical wall (52) and a floor (53). A resilient seal (54) having a central opening (55) is attached at its circumferential edge (56) to the wall (52). The housing (51) has an internal partial wall (57) that is a circular wall generally concentric with the housing wall (51). A flexible, non-resilient sheet (58) shaped like a ring or like a cone with a central hole is provided by attachment to both the cannula flange (50) and the partial wall (57). During a surgical procedure when an instrument shaft (59) is inserted through the opening (55), insufflation pressure and gas travel along the path shown by arrows (60). The maximum diameter of the cannula flange (50) is less than the inside diameter formed by the partial wall (57) so that lateral movement (X-Z plane) of the housing (51) relative to the cannula (48) is permitted. The overlap of the flange (50) and the floor (53) limits upward movement (Y-axis) of the housing (51) relative to the cannula (48). The sheet (58) is sized toe allow desired lateral movement of the housing (51) relative to the cannula (48), but to limit downward (Y-axis) movement of the housing (51) relative to the cannula (48) such as when downward force from the insertion of an instrument through the central opening (55) is applied.

FIG. 6 illustrates yet another embodiment which is similar to that shown in FIG. 5, except that the sheet (61) is attached to the cannula at a different location. For illustration, a drawing line (73) bisects the assembly. A cannula (62) has a proximal end (63) and a flange (64). A seal housing (65) has a vertical wall (66) and a floor (67). A resilient seal (68) having a central opening (69) is attached at its circumferential edge (70) to the wall (66). The housing (65) has an internal partial wall (68) that is a circular wall generally concentric with the housing wall (66). A flexible, non-resilient sheet (69) shaped like a ring or like a cone with a central hole is provided by attachment to both the cannula proximal end (63) and the partial wall (68). During a surgical procedure when an instrument shaft (71) is inserted through the opening (69), insufflation pressure and gas travel along the path shown by arrows (72). The maximum diameter of the cannula flange (64) is less than the inside diameter formed by the partial wall (68) so that lateral movement (X-Z plane) of the housing (65) relative to the cannula (62) is permitted. The overlap of the flange (64) and the floor (67) limits upward movement (Y-axis) of the housing (65) relative to the cannula (62). The sheet (69) is sized toe allow desired lateral movement of the housing (65) relative to the cannula (62), but to limit downward (Yaws) movement of the housing (65) relative to the cannula (62) such as when downward force from the insertion of an instrument through the central opening (69) is applied.

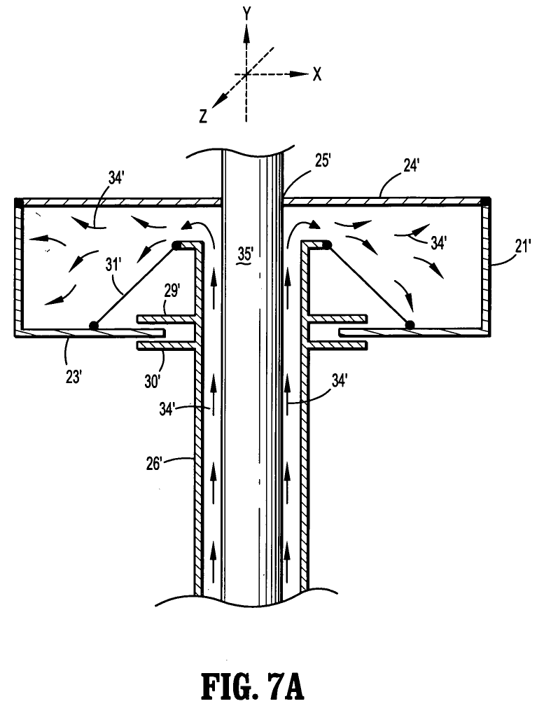

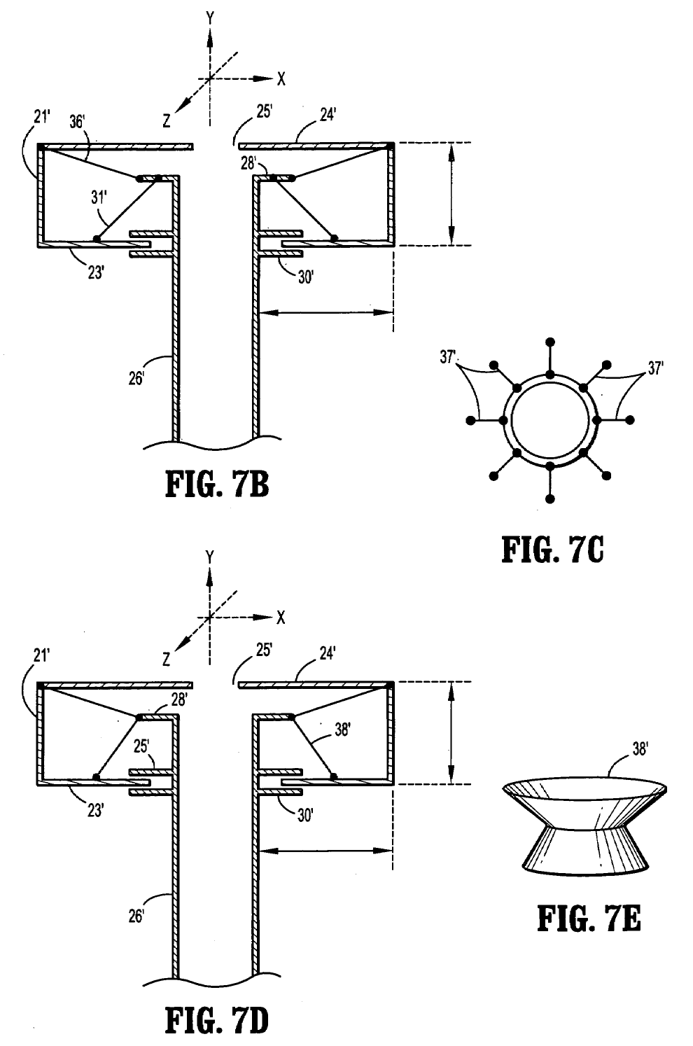

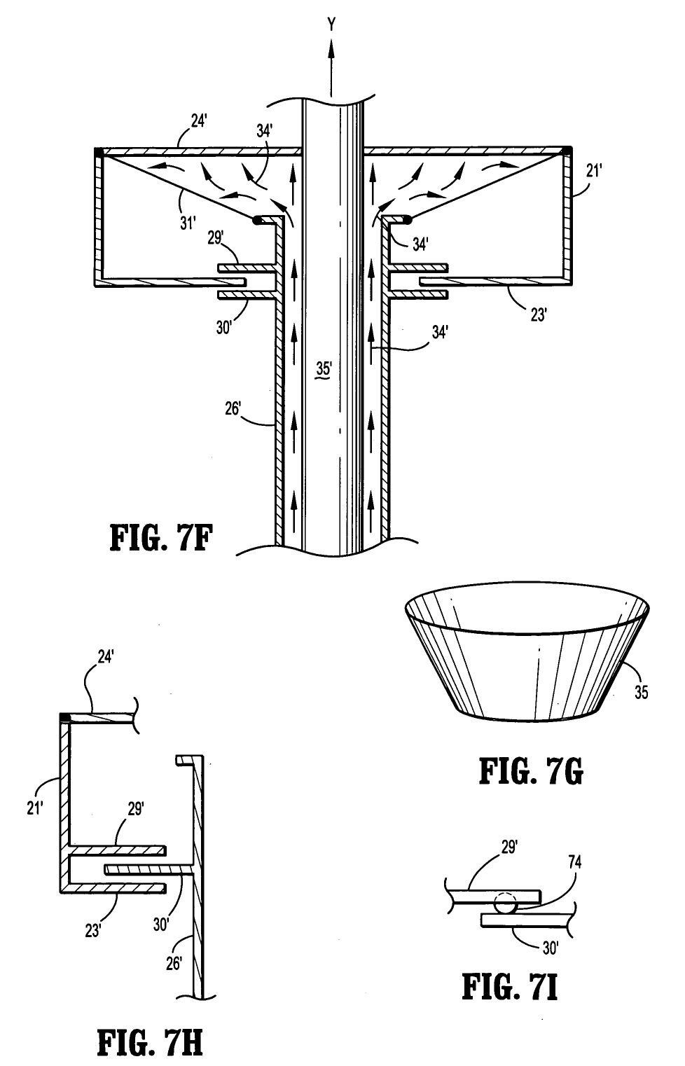

FIGS. 7A and 7B illustrate another embodiment that is similar to FIG. 3 except that the sheet (31′) is attached to the housing (21′) at a different location. Using like numerals as those of FIG. 3, followed with a “′” mark, a housing (21′) comprises a vertical wall (22′) and a lower flange (23′). A resilient seal (24′) is provided having a central opening (25′) and it is attached to the wall (22′) at its circumferential edge. A cannula (26′) has a proximal end (27′) which may have an end flange (28′), and two intermediate flanges (29′, 30′) spaced apart vertically and capturing the lower flange (23′) of the housing (21′) to limit its range of vertical movement relative to the cannula (26′) in the Y-direction shown. The inside diameter of the lower flange (23) is greater than that of the cannula (26′) but less than the maximum diameter of the intermediate flanges (29′, 30′) in order to allow the housing (21′) to move laterally in the X-Z plane. A non-resilient sheet (31′) having a ring-shape or shaped like a cone with a central opening is provided by attaching its inside diameter edge (32′) to the cannula flange (28′) and by attaching its outside circumferential edge (33′) to the inside of the floor (23′). The sheet (31′) may be slightly oversized to enable the housing (21′) to move laterally (X-Z plane) and vertically (Y-axis) relative to the cannula without substantial resistance. The sheet (31′) material is sufficient to seal pressures and gases used during insulation in surgical procedures. It may be made of any suitable material such as those described above with respect to an earlier embodiment. For illustrative purposes, pressure and gas flow arrow (34′) are shown in FIG. 7, indicating the path of pressure and gas during insufflation in a surgical procedure when an instrument shaft (35′) is inserted into the central opening (25′) and the cannula (26′) is introduced into a patient so that its upper, or proximal end is exposed and its distal end is beneath the patients skin.

FIG. 7B illustrates the optional addition of another sheet (36′) to the embodiment of FIG. 7A. The additional sheet (36′) in combination with the other sheet (31′) provides limits on vertical motion (Y-axis) that the housing (21′) may undergo. The additional sheet (36′) may be a sealing sheet such as the first sheet (31′), or it may merely serve a tether function and thus be meshed or even comprised of a series of cords (37′, FIG. 7C) instead of a sheet. Alternatively, the same can be true of the first sheet (31′) which then requires that the additional sheet (36′) function as a seal. Both sheets (31′) and (36′) may be seals to provide a degree of backup.

FIGS. 7D and 7E illustrate another variation of the embodiment shown in FIG. 7A, wherein instead of one sheet (31′) or two (31′, 36′) as shown in FIG. 7B, an hourglass shape sheet (38′) may be used and positioned as shown to provide redundant sealing and enhanced motion limiting over the relative movement of the housing (21′) relative to the cannula (26′).

FIGS. 7F-7G illustrate another variation of the embodiment shown in FIG. 7A, wherein the sheet (31′) is attached to the upper end of the wall (21′). In this variation, the sheet (31′) is inverted as compared to FIG. 7A, as shown in FIG. 7G.

If desired, the arrangement of the cannula flanges (29′, 30′) and the floor (23′) can be reconfigured as shown in FIG. 7H, where an upper flange (29) extends from the housing wall (21′) so that it and the floor (23′) vertically trap a single flange (30′) extending from the cannula (26′).

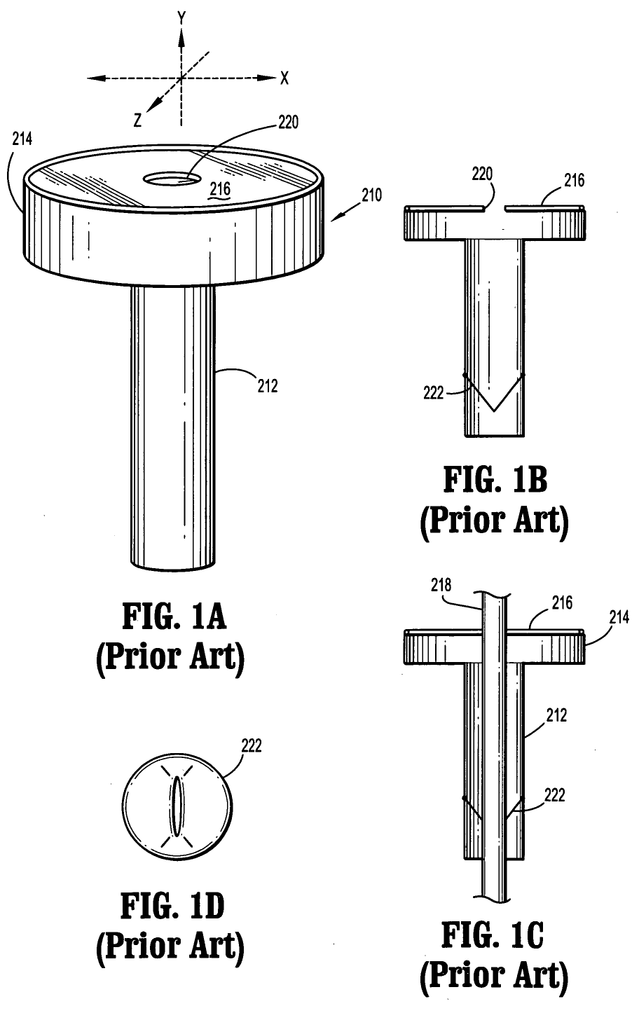

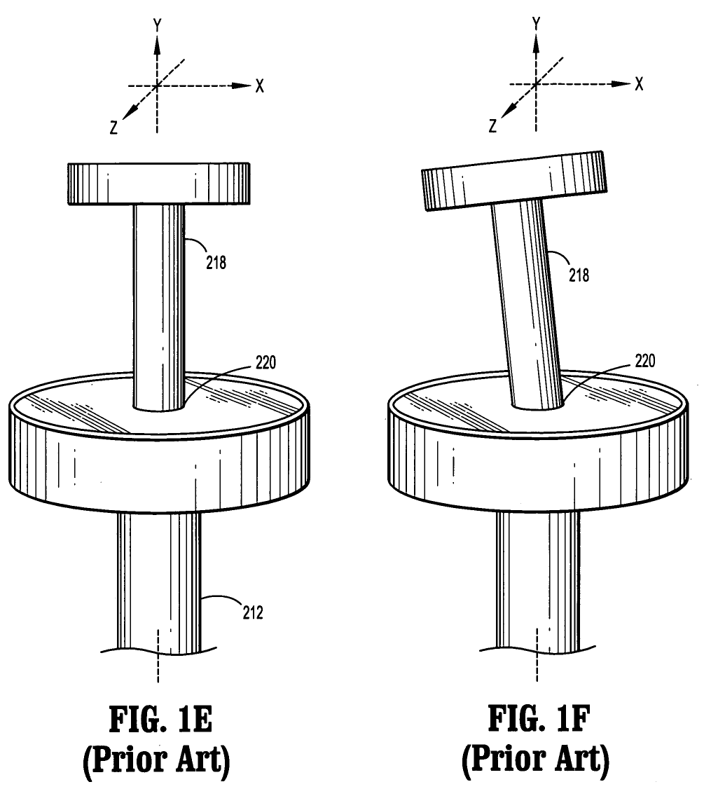

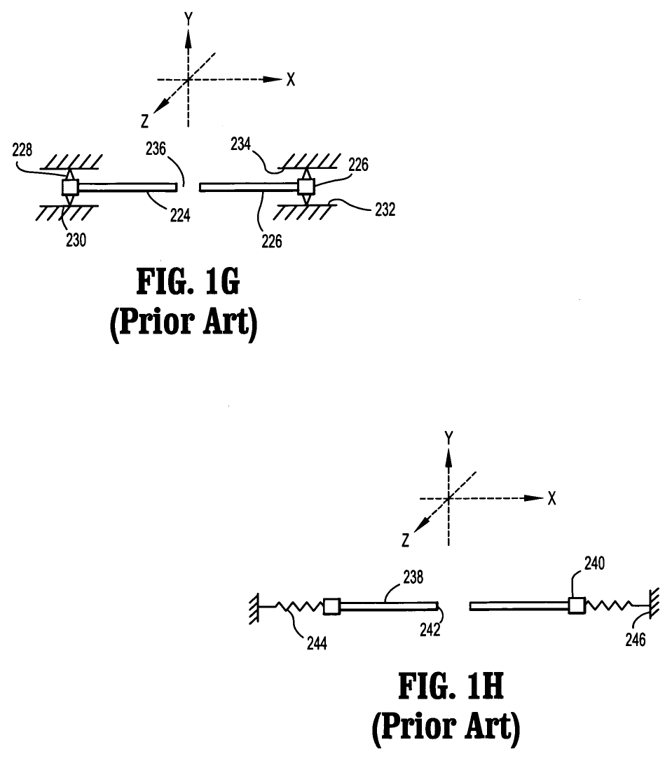

FIG. 1 illustrates the use of ball bearings (74) between flanges (29′, 30′) as one example of different possible ways of reducing friction when the housing moves laterally (X-Z p[lane) to the cannula. Other possibilities include molded or unitary bearing surfaces, raised bumps that minimize contact surface area, and conventional bearing arrangements.

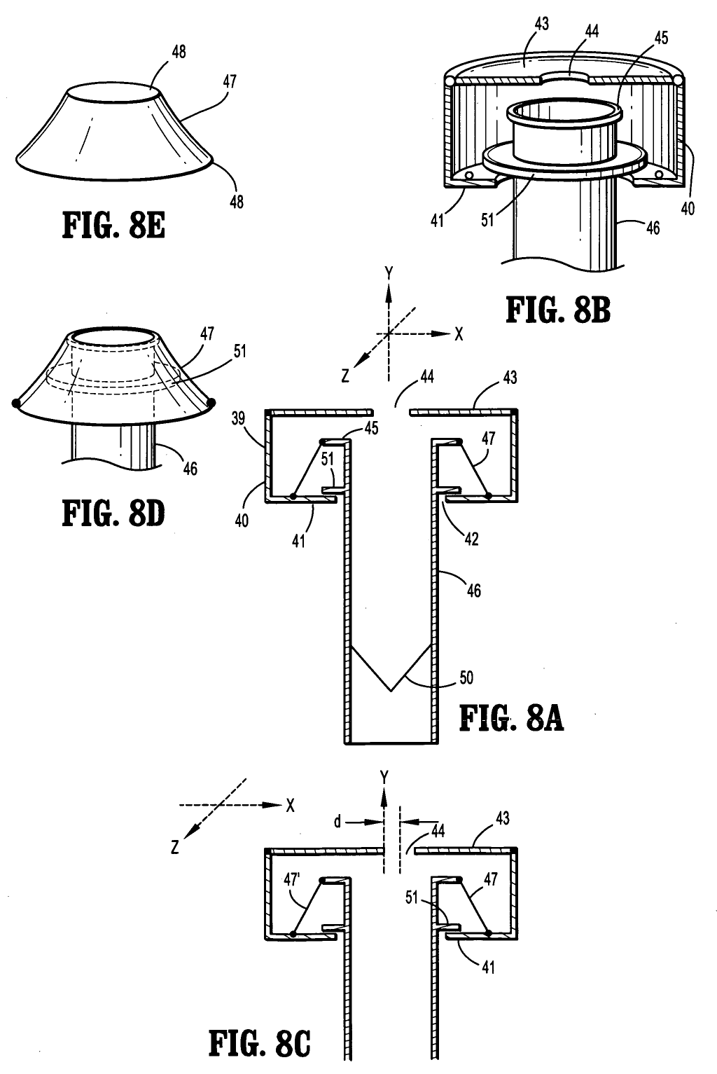

FIGS. 8A-8D illustrate another embodiment of the present invention. A housing (39) comprises a circumferential wall (40), a floor (41) having a central opening (42) therein, and a resilient seal (43) having a central opening (44) therein. The housing (39) is suspended from the proximal end (45) of a cannula (46) by a non-elastic sheet (47). The sheet (47) is preferably shaped like an inverted cone having a central opening (48) and is attached at the opening (48) to the cannula proximal end (45) and is attached at its outside circumferential edge (49) to the floor (41) of the housing (39). A secondary seal (50) of a conventional type is provided in the distal portion of the cannula (46). A flange (51) provided toward the proximal end of the cannula (46) limits upward movement of the housing (39) relative to the cannula (46) because the flange (51) will contact the floor (41) due to their relative configurations. The floor opening (42) has a minimum diameter that is greater than the maximum diameter of the cannula (46) to that the housing (39) may be relatively displaced in a lateral plane, X-Z, relative to the cannula (46) as shown in FIG. 8C. The sheet (47) flexes on the side (47′) where the housing (39) is moved toward the cannula (46) by a distance d from the Y-axis. Because of the location of the attachments of the sheet (47) to the cannula (46) and to the housing (39), respectively, the housing (39) is suspended from and moves relative to the cannula (46) in a pendulum like manner.

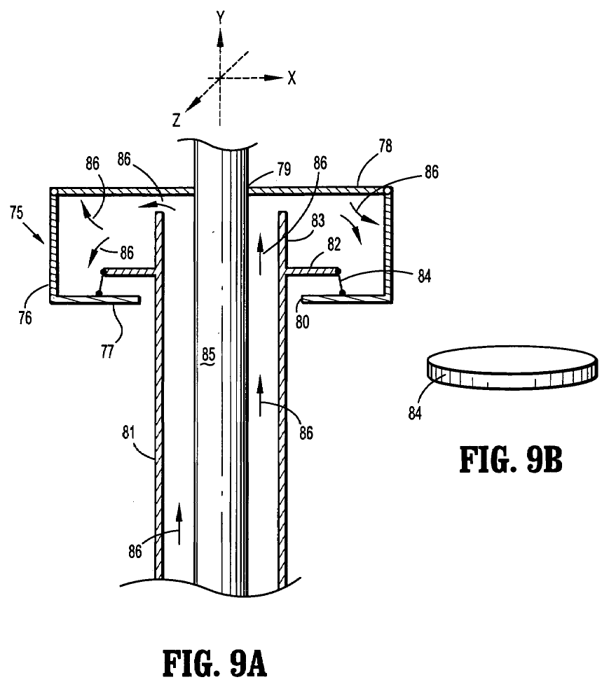

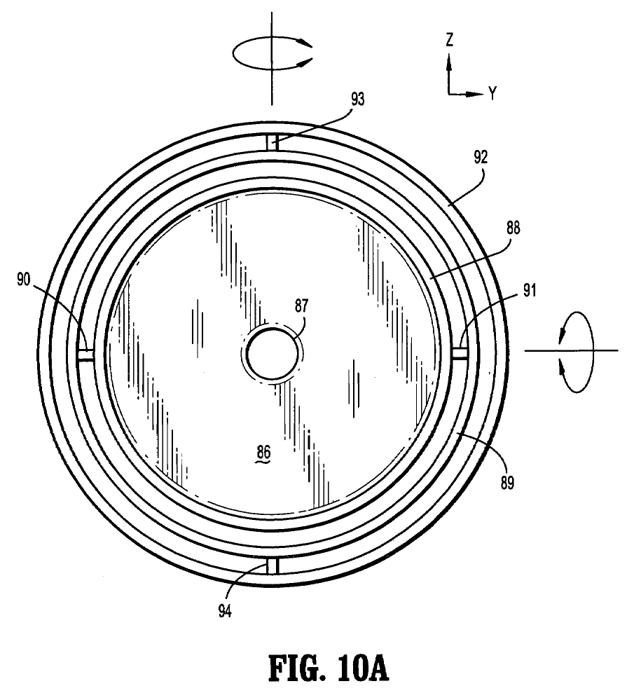

FIGS. 9A-9B illustrate another embodiment of the present invention. A housing (75) has a circumferential wall (6) and a floor (77) having a central opening (80) therein. A resilient seal (78) having a central opening (9) is mounted to the wall (76) at its circumferential edge. A cannula (81) has a flange (82) positioned toward its distal end (83), but not fully reaching the distal end (83). A tube of (84) sheet-like, non-resilient material suspends the housing (75) from the flange (82) as shown in FIG. 9A. When a surgical instrument shaft (85) is inserted through the central seal opening (79), insufflation pressure and gas will travel as indicated by the arrows (86). Lateral movement (X-Z plane) of the housing (5) relative to the cannula (81) is made possible by the minimum diameter of the opening (80) being greater than the outside diameter of the cannula (81). Upward vertical movement (Y-axis) of the housing (75) relative to the cannula (81) is limited by contact between the flange (82) and the floor (77), and downward vertical movement is limited by the length of the tube (84).

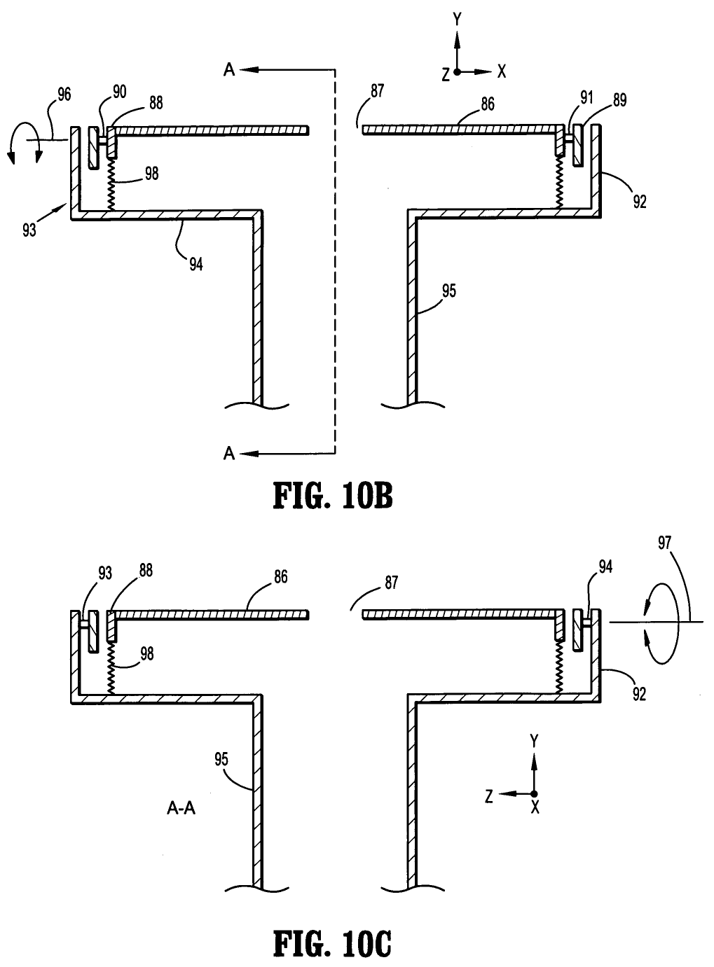

FIGS. 10A-10C illustrate another embodiment of the present invention. A generally round resilient seal (86) having a central opening (87) is mounted at its circumferential edge to a first rigid ring (88). The first rigid ring (88) is pivotally mounted to and concentrically inside of a second ring (89) by two aligned first hinge pins (90, 91) that are on opposite sides of the ring (88). The second ring (89) is pivotally mounted to and concentrically inside of a cylindrical wall (92) by two aligned second hinge pins (93, 94). As such, the seal (86) and the first rigid ring (88) pivot relative to the second ring (89) about an axis defined by the pins (90, 91), while the second ring (89) pivots relative to the cylindrical wall (92) about an axis defined by the pins (93, 94), where the second defined axis (93, 94) is perpendicular to the first defined axis (90, 91). Pivoting of the seal and rings about these two axes as defined provides relative universal pivoting of the seal (86) and its opening (87) relative to the cylindrical wall (92).

In the preferred embodiment shown, the cylindrical wall (92) is part of a housing (93) comprising the wall (92) and a floor (94) connected to the distal end of a cannula (95). Alternatively, the cylindrical wall (92) could simply by the cannula (95) itself.

FIG. 10B provides a front, cross sectional view of the assembly shown in FIG. 10A, and FIG. 1C provides a side, cross sectional view taken at line A-A marked in FIG. 10B.

A tube (98) of non-resilient sheet material is provided between the first ring (88) and the floor (94) to contain pressure and gas from insufflation as it travels up inside the cannula (95) and is trapped beneath the seal (86) when an instrument shaft resides in the opening (87).

In use, the seal (86) will be pivotable about the first axis (96), or the X-axis. The seal (86) will also be pivotable about the second axis (97), or the Z-axis, wherein the Z-axis in FIG. 10B is orthogonal to the page as it is viewed, and in FIG. 10C the axis is orthogonal to the page as it is viewed. The tube (98) will have sufficient slack to accommodate the universal pivoting of the seal (86) and the rings relative to the cylindrical wall (92).

The pivot pins (90, 91 and 93, 94) may be made of a flexible material, such as plastic, so that while they have sufficient rigidity to maintain the pivoting operation herein described, they can bend when axial force is applied relative to them. In this manner, lateral movement (X-Z plane) of the seal (86) can be achieved in order to alleviate lateral forces applied to the opening (87) due to lateral movement of an instrument shaft inserted in the opening (87) and thus reduce the tendency for cat-eyeing or stretching. Alternatively, the pivot pins may be rigid and inflexible, so that only the universal pivoting is achieved.

The design of FIGS. 10A-10C achieves universal type or compound pivoting in a way that when compared to, for example, a ball-and-socket type joint, presents minimal friction and enables a relatively small amount of materials and space to be used.

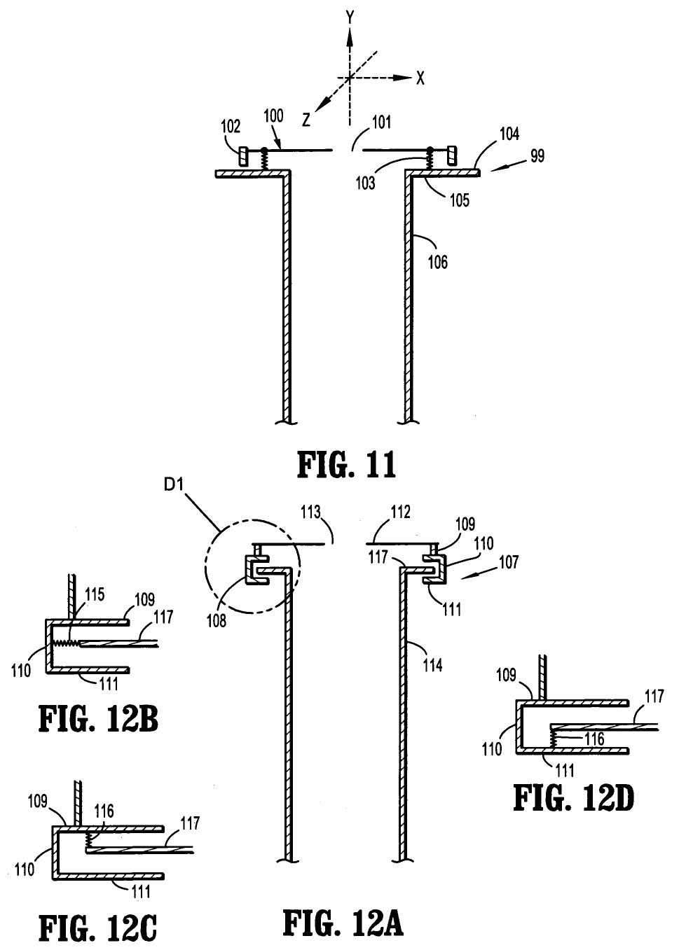

Another embodiment of the present invention is shown in FIG. 11, where an assembly (99) comprises a resilient seal (100) having a central opening (101) therein is mounted to a rigid ring (102). A non-resilient tube (103) of sheet material joins the seal (100) to an upper surface (104) of a flange (105) radially extending from the top edge of a cannula (106). In use, when an instrument shaft is inserted into the opening (101) and manipulated, insufflation pressure and gas from inside the cannula (106) is trapped beneath the seal (100) thereby providing a lifting force thereto. The tube (103) has sufficient slack or length to permit some axial movement of the seal (100) relative to the cannula (106). The tension and therefore length of the tube (103) controls the upward (Y-axis) movement of the seal (100) relative to the cannula (106). The ring (102) has a shape that extends downwardly so that the ring (102) is supported on the flange upper surface (104) when downward force is applied to the seal (100) such as when an instrument is being inserted therein.

FIGS. 12A-12D illustrate another embodiment of the present invention. An assembly (107) according to the present invention comprises a rigid ring (108) having, in cross-section, a u-shape comprising a first leg (109), a second leg (110), and a third leg (111). The ring (108) supports a resilient seal (112) having a central opening (113) therein. The assembly (107) is positioned over the proximal end of a cannula (114) and attached thereto by a non-resilient ring (115, FIG. 12B) or tube (116, FIGS. 12C and 12D). Referring to FIG. 12A, the detailed components referred to by D1 are shown close up in alternative embodiments FIGS. 12B, 12C and 12D. A flange (117) connected to the distal end of the cannula (114) is positioned vertically between first and third legs (109, 111), which are sized to overlap with the flange as shown in FIG. 12A to thereby limit relative vertical movement (Y-axis) of the ring and seal (112) relative to the cannula (114). The non-resilient ring (115) or tube (116), respectively, each seal pressure or gas introduced through the cannula (114) and trapped beneath the seal (112) when an instrument shaft is inserted through the opening (113). The non-resilient ring (115) or tube (116), respectively, are each sized to allow lateral movement (X-Z plane) of the seal (112) relative to the cannula (114), limited by the relative clearance between the minimum diameters of the legs (109, 111) and the outside diameter of the cannula (114).

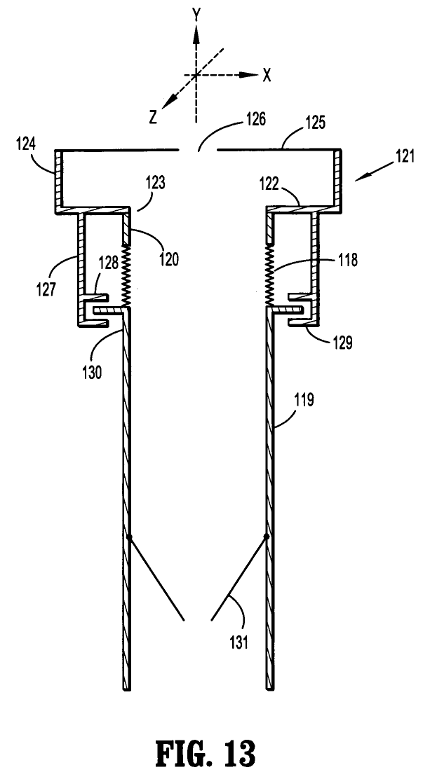

FIG. 13 illustrates another embodiment of the present invention, in which a non resilient, flexible tube (118) joins a rigid cannula (119) to an extending rigid tube portion (120) of a housing (121) comprising a floor (122) having an opening (123) and a cylindrical wall (124). A resilient seal (125) having a central opening (126) is attached at its circumferential edge to the cylindrical wall (124). A downwardly extending cylindrical sub-wall (127) extends from the floor (122) toward the distal end of the cannula (119). A pair of inner flanges (128, 129) spaced apart vertically extend radially inwardly, and their minimum diameters are greater than the outside diameter of the cannula (119). A cannula flange (130) extends radially outwardly from the cannula (119) and is positioned vertically between the inner flanges (128, 129) as shown. Contact between the cannula flange (130) and the lower inner flange (129) limits upward movement of the housing (121) relative to the cannula (119) and contact been the cannula flange (130) and the upper inner flange (128) limits downward movement of the housing (121) relative to the cannula (119). A secondary seal (131) of a type generally known is provided. During use, insufflation pressure and gas from inside the cannula (119) is trapped beneath the seal (125) when an instrument shaft is inserted therein, and is contained by the tube (118). The flexibility of the tube (118) allows the housing (121) and its downward extension (120) to shift laterally (X-Z plane) relative to the cannula (119).

While the preferred embodiments of the present invention have been disclosed, various modification can be made without departing from the scope of the presently claimed invention.

What is claimed is:

1. A surgical seal assembly comprising
a seal having a generally round, flat body and a central opening therein adapted to receive and seal around a shaft;
a first rigid member to which said seal is attached in a sealing manner;
a second rigid member having a passage therethrough and adapted to be inserted a least partially into a patient; and
a non elastic member attached to each of said first rigid member and said second rigid member in a sealing manner and in a manner permitting relative movement of said first rigid member relative to said second rigid member.

2. An assembly according to claim 1, wherein
said first member is adapted to move relative to said second member in a pivoting manner.

3. An assembly according to claim 1, wherein
said seal is resilient.

4. An assembly according to claim 1, wherein
said first member is adapted to move relative to said second member in a linear manner.

5. An assembly according to claim 4, wherein
said first member is adapted to move relative to said second member in a pivoting manner.

6. An assembly according to claim 1, further comprising
a hinge rotatably connecting said first member and said second member about a first hinge axis.

7. An assembly according to claim 6, further comprising
a second hinge rotatably connecting said first member and said second member about a second axis, said second axis being aligned orthogonally with respect to said first axis.
